(19) United States
(12) Patent Application Publication
(10) Pub. No.: US 2010/0179412 A1
(43) Pub. Date: Jul. 15, 2010

(54) ARRANGEMENT FOR MAGNETIC PARTICLE IMAGING, METHOD FOR INFLUENCING AND/OR DETECTING MAGNETIC PARTICLES AND MAGNETIC PARTICLE

(75) Inventors: Jurgen Weizenecker, Eindhoven (NL); Bernhard Gleich, Eindhoven (NL); Hans Negle, Eindhoven (NL); Arne Lunding, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/526,554

(22) PCT Filed: Feb. 11, 2008
(86) PCT No.: PCT/IB2008/050490

§ 371 Date: Mar. 4, 2010

(30) Foreign Application Priority Data
Feb. 15, 2007 (EP) 07102442.6

(51) Int. Cl. A61B 5/05

(52) U.S. Cl. 600/409

(57) ABSTRACT

An arrangement for magnetic particle imaging and a method for influencing and/or detecting magnetic particles in a region of action is disclosed, which arrangement comprises: magnetic particles in a region of action, the magnetic particles being influenceable and/or detectable, selection means for generating a magnetic selection field having a pattern in space of its magnetic field strength such that a first sub-zone having a low magnetic field strength and a second sub-zone having a higher magnetic field strengthare formed in the region of action, drive means for changing the position in space of the two sub-zones in the region of action by means of a magnetic drive field so that the magnetization of the magnetic particles changes locally, wherein each magneticparticle comprises a non-magnetic substrate with a layer of stainless steel.

The present invention relates to an arrangement for magnetic particle imaging. Furthermore, the invention relates to a method for influencing and/or detecting magnetic particles in a region of action and to a magnetic particle for use in such an arrangement and/or in such a method.

The arrangement and the method of this kind is known from German patent application DE 101 51 778 A1. In the case of the method described in that publication, first of all a magnetic field having a spatial distribution of the magnetic field strength is generated such that a first sub-zone having a relatively low magnetic field strength and a second sub-zone having a relatively high magnetic field strength are formed in the examination zone. The position in space of the sub-zones in the examination zone is then shifted, so that the magnetization of the particles in the examination zone changes locally. Signals are recorded which are dependent on the magnetization in the examination zone, which magnetization has been influenced by the shift in the position in space of the sub-zones, and information concerning the spatial distribution of the magnetic particles in the examination zone is extracted from these signals, so that an image of the examination zone can be formed. Such an arrangement and such a method have the advantage that it can be used to examine arbitrary examination objects—e.g. human bodies—in a non-destructive manner and without causing any damage and with a high special resolution, both close to the surface and remote from the surface of the examination object.

In known arrangements of this type the performance of the tracer material is essential for the performance of the whole method. It is a drawback of non-mono-domain particles that their performance is not sufficient, as the magnetic field ‘seen’ by the particles (or domains) are dominated by the demagnetization fields. In consequence this means, that at low applied magnetic fields the magnetization changes only linearly with the external magnetic field.

It is therefore an objective of the present invention to provide an arrangement and a method of the kind mentioned initially, where non-mono-domain magnetic particles with a reduced demagnetization factor are used.

The above objective is achieved by an arrangement for magnetic particle imaging, which arrangement comprises:

magnetic particles in a region of action, the magnetic particles being influenceable and/or detectable,

selection means for generating a magnetic selection field having a pattern in space of its magnetic field strength such that a first sub-zone having a low magnetic field strength and a second sub-zone having a higher magnetic field strength are formed in the region of action,

drive means for changing the position in space of the two sub-zones in the region of action by means of a magnetic drive field so that the magnetization of the magnetic particles changes locally,

wherein each magnetic particle comprises a non-magnetic substrate with a layer of stainless steel.

The above objective is also achieved by a method for influencing and/or detecting magnetic particles in a region of action, wherein the method comprises the steps of

introducing magnetic particles into a region of action, each magnetic particle comprising a non-magnetic substrate with a layer of stainless steel,

generating a magnetic selection field having a pattern in space of its magnetic field strength such that a first sub-zone having a low magnetic field strength and a second sub-zone having a higher magnetic field strength are formed in the region of action,

changing the position in space of the two sub-zones in the region of action by means of a magnetic drive field so that the magnetization of the magnetic particles changes locally.

The inventive arrangement and method according to the present invention have the advantage that the magnetic particles provide a surprisingly high signal strength. The demagnetization factor of the stainless steel coated non-magnetic substrate is advantageously lower than that of a massive magnetic substrate of the same dimension.

According to the present invention, it is to be understood that the selection means and/or the drive means and/or the receiving means can at least partially be provided in the form of one single coil or solenoid. However, it is preferred according to the present invention that separate coils are provided to form the selection means, the drive means and the receiving means. Furthermore, the selection means can comprise one or more permanent magnets located more distant from the region of action than the drive means. Furthermore according to the present invention, the selection means and/or the drive means and/or the receiving means can each be composed of separate individual parts, especially separate individual coils or solenoids, provided and/or arranged such that the separate parts form together the selection means and/or the drive means and/or the receiving means. Especially for the drive means and/or the selection means, a plurality of parts, especially pairs for coils (e.g. in a Helmholtz or Anti-Helmholtz configuration) are preferred in order to provide the possibility to generate and/or to detect components of magnetic fields directed in different spatial directions.

Another object of the present invention is a magnetic particle for use in an arrangement according to the invention and/or for use in a method according to the invention, the magnetic particle comprising a non-magnetic substrate with a layer of stainless steel.

The magnetic particle is advantageous in production, regarding material costs and/or production process. Stainless steel, in the sense of the present invention has a higher resistance to oxidation and corrosion in many natural and man made environments, than other steel. In particular, according to the invention, stainless steel is a ferrous alloy with a minimum of 10.5% chromium. Preferably, the magnetic particle does not comprise any protective coating, as the stainless steel layer is advantageously resistive by itself.

According to the present invention, it is preferred that the layer of stainless steel is weak magnetic. Advantageously, agglomeration of the particles is prevented by using weak magnetic stainless steel. Weak magnetic, in the sense of the invention, means that a value of a saturation magnetization is below 0.8 Tesla. Preferably, the value of the saturation magnetization is between 0.1 Tesla and 0.6 Tesla. The saturation magnetization is specified in Tesla which is not fully correct in the sense of the International System of Units (SI). to obtain the correct values, a division by the magnetic field constant μ₀, has to be done, as Tesla is the unit of the magnetic flux density.

It is furthermore preferred according to the present invention that the substrate is spherical. More preferably the substrate is a glass substrate.

It is furthermore preferred according to the present invention that a diameter of the substrate is at least 1000 times larger than a thickness of the layer, more preferably the diameter of the substrate is at least 10000 times larger than the thickness of the layer. Advantageously, the demagnetization factor of the magnetic particle can be reduced by increasing the ratio between the substrate diameter and the layer thickness.

Preferred examples of a stainless steel alloys the layer is made of, comprise at least one of the elements nickel, manganese, molybdenum, copper and niobium.

According to the present invention, it is preferred that the stainless steel alloy comprises chromium, in particular between, 10.5% and 20% by weight, more preferable between 14% and 16% by weight.

Furthermore, it is preferred that the stainless steel alloy comprises nickel, in particular between 5% and 15% by weight, more preferable between 8% and 12% by weight.

Furthermore, it is preferred that the stainless steel alloy comprises manganese, preferably between, 0.5% and 4% by weight, more preferable between 1.5% and 2.5% by weight.

These and other characteristics, features and advantages of the present invention will become apparent from the following detailed description, taken in conjunction with the accompanying drawings, which illustrate, by way of example, the principles of the invention. The description is given for the sake of example only, without limiting the scope of the invention. The reference figures quoted below refer to the attached drawings.

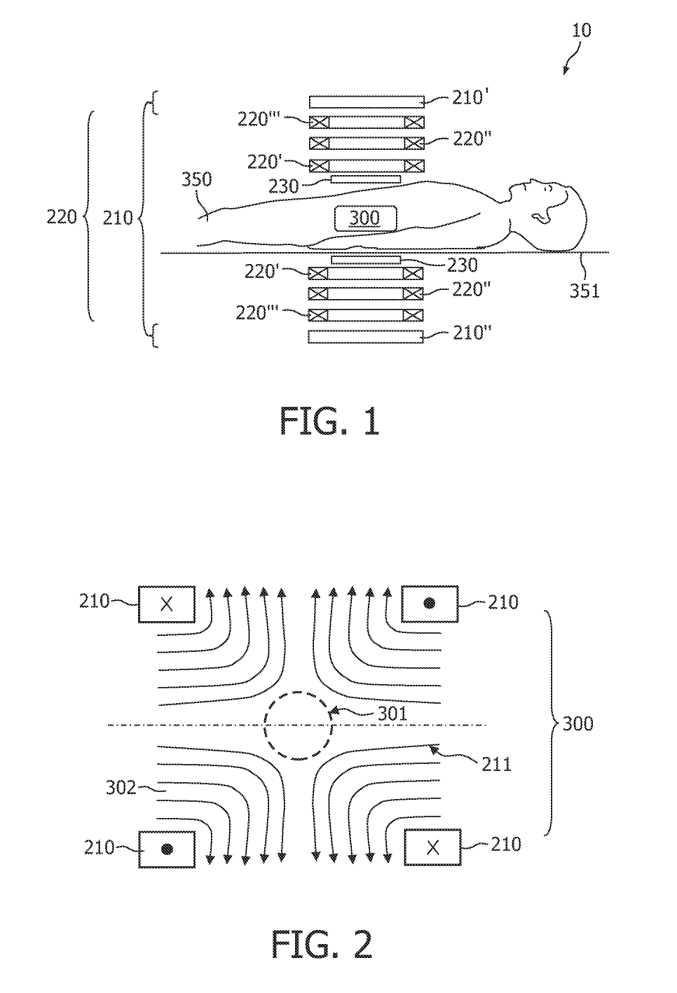

FIG. 1 illustrates an arrangement according to the present invention for carrying out the method according to the present invention.

FIG. 2 illustrates an example of the field line pattern produced by an arrangement according to the present invention

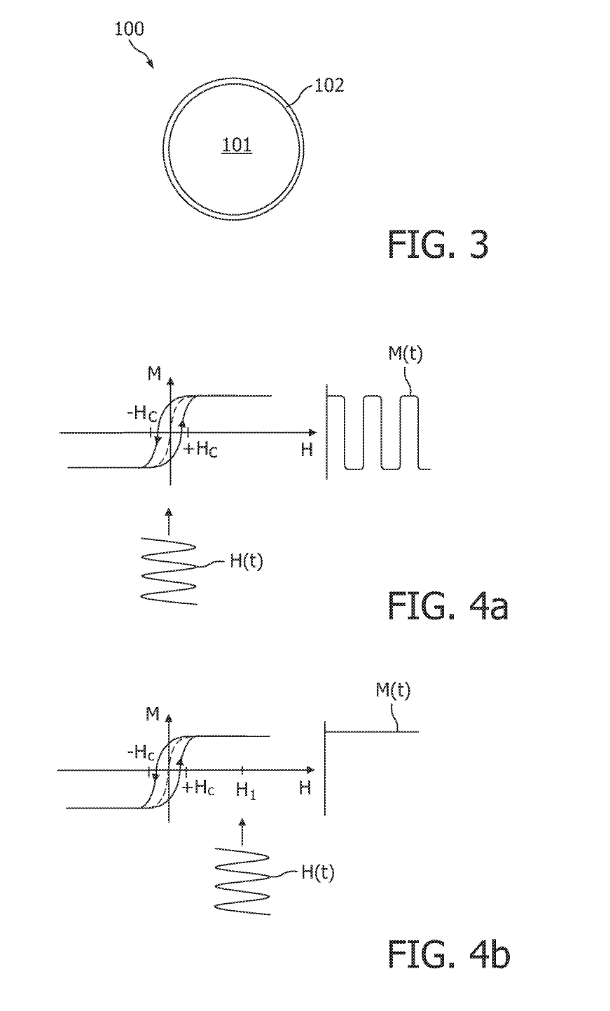

FIG. 3 illustrates an enlarged view of a magnetic particle present in the region of action.

FIGS. 4a and 4b illustrate the magnetization characteristics of such particles.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes.

Where an indefinite or definite article is used when referring to a singular noun, e.g. “a”, “an”, “the”, this includes a plural of that noun unless something else is specifically stated.

Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described of illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the present description and claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

In FIG. 1, an arbitrary object to be examined by means of an arrangement 10 according to the present invention is shown. The reference numeral 350 in FIG. 1 denotes an object, in this case a human or animal patient, who is arranged on a patient table, only part of the top of which is shown. Prior to the application of the method according to the present invention, magnetic particles 100 (not shown in FIG. 1) are arranged in a region of action 300 of the inventive arrangement 10. Especially prior to a therapeutical and/or diagnostical treatment of, for example, a tumor, the magnetic particles 100 are positioned in the region of action 300, e.g. by means of a liquid (not shown) comprising the magnetic particles 100 which is injected into the body of the patient 350.

As an example of an embodiment of the present invention, an arrangement 10 is shown in FIG. 2 comprising a plurality of coils forming a selection means 210 whose range defines the region of action 300 which is also called the region of treatment 300. For example, the selection means 210 is arranged above and below the patient 350 or above and below the table top. For example, the selection means 210 comprise a first pair of coils 210′, 210″, each comprising two identically constructed windings 210′ and 210″ which are arranged coaxially above and below the patient 350 and which are traversed by equal currents, especially in opposed directions. The first coil pair 210′, 210″ together are called selection means 210 in the following. Preferably, direct currents are used in this case.

The selection means 210 generate a magnetic selection field 211 which is in general a gradient magnetic field which is represented schematically in FIG. 2 by the field lines. It has a substantially constant gradient in the direction of the (e.g. vertical) axis of the coil pair of the selection means 210 and reaches the value zero in a point on this axis. Starting from this field-free point (not individually shown in FIG. 2), the field strength of the magnetic selection field 211 increases in all three spatial directions as the distance increases from the field-free point. In a first sub-zone 301 or region 301 which is denoted by a dashed line around the field-free point the field strength is so small that the magnetization of particles 100 present in that first sub-zone 301 is not saturated, whereas the magnetization of particles 100 present in a second sub-zone 302 (outside the region 301) is in a state of saturation. The field-free point or first sub-zone 301 of the region of action 300 is preferably a spatially coherent area; it may also be a punctiform area or else a line or a flat area. In the second sub-zone 302 (i.e. in the residual part of the region of action 300 outside of the first sub-zone 301) the magnetic field strength is sufficiently strong to keep the particles 100 in a state of saturation. By changing the position of the two sub-zones 301, 302 within the region of action 300, the (overall) magnetization in the region of action 300 changes. By measuring the magnetization in the region of action 300 or a physical parameters influenced by the magnetization, information about the spatial distribution of the magnetic particles in the region of action can be obtained. In order to change the relative spatial position of the two sub-zones 301, 302 in the region of action 300, a further magnetic field, the so-called magnetic drive field 221, is superposed to the selection field 211 in the region of action 300 or at least in a part of the region of action 300.

FIG. 3 shows an example of a magnetic particle 100 according to the present invention which is used together with an arrangement 10 of the present invention. It comprises a non-magnetic substrate 101 which is, for example, spherical. The non-magnetic substrate 101 is, for example, made of glass. The non-magnetic substrate 101 is provided with a layer 102 of stainless steel which has a thickness of, for example, 5 nm. This layer 102 of stainless steel is not covered by any coating, as the layer 102 itself is advantageously resistive against chemically and/or physically aggressive environments, e.g. acids. The magnetic field strength of the magnetic selection field 211 required for the saturation of the magnetization of such particles 100 is dependent on various parameters, in particular the diameter of the particles 100, the thickness of the stainless steel layer 102 and their ratio. The person skilled in the art will recognise that the depicted particle 100 does not represent the actual ratio of thickness of the layer 102 and the diameter of the substrate 101. In fact, the diameter of the substrate 101 is approximately similar to the diameter of the magnetic particle 100.

In the case of e.g. a diameter of the substrate 101 (or magnetic particle 100) of 10 μm, a magnetic field of approximately 800 A/m (corresponding approximately to a flux density of 1 mT) is then required, whereas in the case of a diameter of 100 μm a magnetic field of 80 A/m suffices. Even smaller values may be obtained when the thickness of the stainless steel layer 102 is reduced.

The size of the first sub-zone 301 is dependent on the one hand on the strength of the gradient of the magnetic selection field 211 and on the other hand on the field strength of the magnetic field required for saturation. For a sufficient saturation of the magnetic particles 100 at a magnetic field strength of 80 A/m and a gradient (in a given space direction) of the field strength of the magnetic selection field 211 amounting to 160·10³ A/m², the first sub-zone 301 in which the magnetization of the particles 100 is not saturated has dimensions of about 1 mm (in the given space direction). By increasing the magnetic field strength and especially the magnetic gradient strength of the magnetic selection field 211, is its possible to enhance the spatial resolution of the arrangement 10 according to the present invention.

When a further magnetic field—in the following called a magnetic drive field 221 is superposed on the magnetic selection field 210 (or gradient magnetic field 210) in the region of action 300, the first sub-zone 301 is shifted relative to the second sub-zone 302 in the direction of this magnetic drive field 221; the extent of this shift increases as the strength of the magnetic drive field 221 increases. When the superposed magnetic drive field 221 is variable in time, the position of the first sub-zone 301 varies accordingly in time and in space. It is advantageous to receive or to detect signals from the magnetic particles 100 located in the first sub-zone 301 in another frequency band (shifted to higher frequencies) than the frequency band of the magnetic drive field 221 variations. This is possible because frequency components of higher harmonics of the magnetic drive field 221 frequency occur due to a change in magnetization of the magnetic particles 100 in the region of action 300 as a result of the non-linearity of the magnetization characteristics.

In order to generate these magnetic drive fields 221 for any given direction in space, there are provided three further coil pairs, namely a second coil pair 220′, a third coil pair 220″ and a fourth coil pair 220′″ which together are called drive means 220 in the following. For example, the second coil pair 220′ generates a component of the magnetic drive field 221 which extends in the direction of the coil axis of the first coil pair 210′, 210″ or the selection means 210, i.e. for example vertically. To this end the windings of the second coil pair 220′ are traversed by equal currents in the same direction. The effect that can be achieved by means of the second coil pair 220′ can in principle also be achieved by the superposition of currents in the same direction on the opposed, equal currents in the first coil pair 210′, 210″, so that the current decreases in one coil and increases in the other coil. However, and especially for the purpose of a signal interpretation with a higher signal to noise ratio, it may be advantageous when the temporally constant (or quasi constant) selection field 211 (also called gradient magnetic field) and the temporally variable vertical magnetic drive field are generated by separate coil pairs of the selection means 210 and of the drive means 220.

The two further coil pairs 220″, 220′″ are provided in order to generate components of the magnetic drive field 221 which extend in a different direction in space, e.g. horizontally in the longitudinal direction of the region of action 300 (or the patient 350) and in a direction perpendicular thereto. If third and fourth coil pairs 220″, 220′″ of the Helmholtz type (like the coil pairs for the selection means 210 and the drive means 220) were used for this purpose, these coil pairs would have to be arranged to the left and the right of the region of treatment or in front of and behind this region, respectively. This would affect the accessibility of the region of action 300 or the region of treatment 300. Therefore, the third and/or fourth magnetic coil pairs or coils 220″, 220′″ are also arranged above and below the region of action 300 and, therefore, their winding configuration must be different from that of the coil pair second coil pair 220′. Coils of this kind, however, are known from the field of magnetic resonance apparatus with open magnets (open MRI) in which an radio frequency (RF) coil pair is situated above and below the region of treatment, said RF coil pair being capable of generating a horizontal, temporally variable magnetic field. Therefore, the construction of such coils need not be further elaborated herein.

The arrangement 10 according to the present invention further comprise selection means 230 that are only schematically shown in FIG. 1. The selection means 230 usually comprise coils that are able to detect the signals induced by magnetization pattern of the magnetic particles 100 in the region of action 300. Coils of this kind, however, are known from the field of magnetic resonance apparatus in which e.g. a radio frequency (RF) coil pair is situated around the region of action 300 in order to have a signal to noise ratio as high as possible. Therefore, the construction of such coils need not be further elaborated herein.

The frequency ranges usually used for or in the different components of the selection means 210, drive means 220 and receiving means 230 are roughly as follows: The magnetic field generated by the selection means 210 does either not vary at all over the time or the variation is comparably slow, preferably between approximately 1 Hz and approximately 100 Hz. The magnetic field generated by the drive means 220 varies preferably between approximately 25 kHz and approximately 100 kHz. The magnetic field variations that the receiving means are supposed to be sensitive are preferably in a frequency range of approximately 50 kHz to approximately 10 MHz.

FIGS. 4a and 4b show the magnetization characteristic, that is, the variation of the magnetization M of a particle 100 (not shown in FIGS. 4a and 4b) as a function of the field strength H at the location of that particle 100, in a dispersion with such particles. It appears that the magnetization M no longer changes beyond a field strength +H_{c} and below a field strength −H_{c}, which means that a saturated magnetization is involved. The magnetization M is not saturated between the values +H_{c} and −H_{c}.

FIG. 4a illustrates the effect of a sinusoidal magnetic field H(t) at the location of the particle 100 where the absolute values of the resulting sinusoidal magnetic field H(t) (i.e. “seen by the particle 100”) are lower than the magnetic field strength required to magnetically saturate the particle 100, i.e. in the case where no further magnetic field is active. The magnetization of the particle 100 or particels 100 for this condition reciprocates between its saturation values at the rhythm of the frequency of the magnetic field H(t). The resultant variation in time of the magnetization is denoted by the reference M(t) on the right hand side of FIG. 4a. It appears that the magnetization also changes periodically and that the magnetization of such a particle is periodically reversed.

The dashed part of the line at the centre of the curve denotes the approximate mean variation of the magnetization M(t) as a function of the field strength of the sinusoidal magnetic field H(t). As a deviation from this centre line, the magnetization extends slightly to the right when the magnetic field H increases from −H_{c} to +H_{c} and slightly to the left when the magnetic field H decreases from +H_{c} to −H_{c}. This known effect is called a hysteresis effect which underlies a mechanism for the generation of heat. The hysteresis surface area which is formed between the paths of the curve and whose shape and size are dependent on the material, is a measure for the generation of heat upon variation of the magnetization.

FIG. 4b shows the effect of a sinusoidal magnetic field H(t) on which a static magnetic field H₁ is superposed. Because the magnetization is in the saturated state, it is practically not influenced by the sinusoidal magnetic field H(t). The magnetization M(t) remains constant in time at this area. Consequently, the magnetic field H(t) does not cause a change of the state of the magnetization.

1. An arrangement (10) for magnetic particle imaging, which arrangement comprises:
magnetic particles (100) in a region of action (300), the magnetic particles being influenceable and/or detectable,
selection means (210) for generating a magnetic selection field (211) having a pattern in space of its magnetic field strength such that a first sub-zone (301) having a low magnetic field strength and a second sub-zone (302) having a higher magnetic field strength are formed in the region of action (300),
drive means (220) for changing the position in space of the two sub-zones (301, 302) in the region of action (300) by means of a magnetic drive field (221) so that the magnetization of the magnetic particles (100) changes locally,
wherein each magnetic particle (100) comprises a non-magnetic substrate (101) with a layer (102) of stainless steel.

2. A method for influencing and/or detecting magnetic particles (100) in a region of action (300), wherein the method comprises the steps of
introducing magnetic particles (100) into a region of action (300), each magnetic particle (100) comprising a non-magnetic substrate (101) with a layer (102) of stainless steel,
generating a magnetic selection field (211) having a pattern in space of its magnetic field strength such that a first sub-zone (301) having a low magnetic field strength and a second sub-zone (302) having a higher magnetic field strength are formed in the region of action (300),
changing the position in space of the two sub-zones (301, 302) in the region of action (300) by means of a magnetic drive field (221) so that the magnetization of the magnetic particles (100) changes locally.

3. Magnetic particle (100) for use in an arrangement (10) according to claim 1, the magnetic particle (100) comprising a non-magnetic substrate (101) with a layer (102) of stainless steel.

4. Magnetic particle (100) according to claim 3, wherein the layer (101) of stainless steel is weak magnetic.

5. Magnetic particle (100) according to claim 3, wherein the substrate (101) is spherical.

6. Magnetic particle (100) according to claim 3, wherein a diameter of the substrate (101) is at least 1000 times larger than a thickness of the layer (101), preferably the diameter of the substrate (101) is at least 10000 times larger than the thickness of the layer (101).

7. Magnetic particle (100) according to claim 3, wherein the layer (101) is made of a stainless steel alloy with at least one of the elements nickel, manganese, molybdenum, copper and niobium.

8. Magnetic particle (100) according to claim 7, wherein the stainless steel alloy comprises chrome, preferably between 10.5% and 20% by weight.

9. Magnetic particle (100) according to claim 7, wherein the stainless steel alloy comprises nickel, preferably between 5% and 15% by weight.

10. Magnetic particle (100)according to claim 7, wherein the stainless steel alloy comprises manganese, preferably between 0.5% and 4% by weight.
